SEQUENCE LISTING (66 sequences)

SEQ ID NO: 1 - protein, 10 aa, Artificial Sequence
  CDRH1
GYSFTSYWIG

SEQ ID NO: 2 - protein, 10 aa, Artificial Sequence
  CDRH2
VIYPGDSYTR

SEQ ID NO: 3 - protein, 9 aa, Artificial Sequence
  CDRH3
  MISC_FEATURE (7)..(7): Xaa is Phe or Leu
  MISC_FEATURE (9)..(9): Xaa is His, Tyr, Thr, or Ser
MPNWGSXDX

SEQ ID NO: 4 - protein, 9 aa, Artificial Sequence
  CDRH3
MPNWGSLDH

SEQ ID NO: 5 - protein, 12 aa, Artificial Sequence
  CDRL1
RASQSISSSYLA

SEQ ID NO: 6 - protein, 7 aa, Artificial Sequence
  CDRL2
GASSRAT

SEQ ID NO: 7 - protein, 5 aa, Artificial Sequence
  CDRL3
QQYAS

SEQ ID NO: 8 - protein, 118 aa, Artificial Sequence
  VH region
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARMPNWGSLDHWGQGTLVTVSS

SEQ ID NO: 9 - protein, 104 aa, Artificial Sequence
  VL region
EIVLTQSPGTLSLSPGERATLSCRASQSISSSYLAWYQQKPGQAPRLLIYGASSRATGIPDRFSGSGSGTDFTLTISRLEPEDFAVYYCQQYASFGQGTKVEIK

SEQ ID NO: 10 - protein, 445 aa, Artificial Sequence
  IgG4 heavy chain
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARMPNWGSLDHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 211 aa, Artificial Sequence
  Kappa light chain
EIVLTQSPGTLSLSPGERATLSCRASQSISSSYLAWYQQKPGQAPRLLIYGASSRATGIPDRFSGSGSGTDFTLTISRLEPEDFAVYYCQQYASFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 9 aa, Artificial Sequence
  CDRH3
  MISC_FEATURE (1)..(1): Xaa denotes Phe, Met, Gln, Leu or Val
  MISC_FEATURE (6)..(6): Xaa denotes Ser or Ala
  MISC_FEATURE (7)..(7): Xaa denotes Phe, Leu, Ala or Met
  MISC_FEATURE (9)..(9): Xaa denotes Tyr, Gln, Lys, Arg, Trp, His, Ala, Thr, Ser, Asn or Gly
XPNWGXXDX

SEQ ID NO: 13 - protein, 9 aa, Artificial Sequence
  CDR H3 SEQUENCE
FPNWGALDQ

SEQ ID NO: 14 - protein, 9 aa, Artificial Sequence
  CDR H3 SEQUENCE
VPNMGSLDT

SEQ ID NO: 15 - protein, 9 aa, Artificial Sequence
  CDR H3 SEQUENCE
FPNWGSMDA

SEQ ID NO: 16 - protein, 9 aa, Artificial Sequence
  CDR H3 SEQUENCE
FPNWGSLDH

SEQ ID NO: 17 - protein, 9 aa, Artificial Sequence
  CDR H3 SEQUENCE
MPNWGSFDY

SEQ ID NO: 18 - protein, 9 aa, Artificial Sequence
  CDR H3 SEQUENCE
MPNWGSFDT

SEQ ID NO: 19 - protein, 9 aa, Artificial Sequence
  CDR H3 SEQUENCE
MPNWGSFDS

SEQ ID NO: 20 - protein, 9 aa, Artificial Sequence
  CDR H3 SEQUENCE
MPNWGSLDT

SEQ ID NO: 21 - protein, 9 aa, Artificial Sequence
  CDR H3 SEQUENCE
MPNWGSLDA

SEQ ID NO: 22 - protein, 9 aa, Artificial Sequence
  CDR H3 SEQUENCE
MPNWGSLDN

SEQ ID NO: 23 - protein, 9 aa, Artificial Sequence
  CDR H3 SEQUENCE
MPNWGALDS

SEQ ID NO: 24 - protein, 9 aa, Artificial Sequence
  CDR H3 SEQUENCE
MPNWGSLDN

SEQ ID NO: 25 - protein, 9 aa, Artificial Sequence
  CDR H3 SEQUENCE
MPNWGSLDY

SEQ ID NO: 26 - protein, 9 aa, Artificial Sequence
  CDR H3 SEQUENCE
MPNWGSFDH

SEQ ID NO: 27 - protein, 9 aa, Artificial Sequence
  CDR H3 SEQUENCE
MPNWGSLDS

SEQ ID NO: 28 - protein, 9 aa, Artificial Sequence
  CDR H3 SEQUENCE
MPNWGSLDG

SEQ ID NO: 29 - protein, 9 aa, Artificial Sequence
  CDR H3 SEQUENCE
VPNWGSLDG

SEQ ID NO: 30 - protein, 28 aa, Artificial Sequence
  CDR H3 SEQUENCE
CARFPNWGSLDHWGQGTLVTVSSASIKG

SEQ ID NO: 31 - protein, 28 aa, Artificial Sequence
  CDR H3 SEQUENCE
CARMPNWGSLDHWGQGTLVTVSSASTKG

SEQ ID NO: 32 - protein, 28 aa, Artificial Sequence
  CDR H3 SEQUENCE
CARMPNWGSFDYWGQGTLVTVSSASIKG

SEQ ID NO: 33 - protein, 12 aa, Artificial Sequence
  CDR H3 SEQUENCE
VRMPNWGSLDHW

SEQ ID NO: 34 - protein, 27 aa, Artificial Sequence
  CDR H3 SEQUENCE
VRMPNWGSLDHWGQGTLVTVSSADIKG

SEQ ID NO: 35 - protein, 27 aa, Artificial Sequence
  CDR H3 SEQUENCE
ARMPNWGSLDHWGQGTLVTVSSASIKG

SEQ ID NO: 36 - protein, 25 aa, Artificial Sequence
  CDR H3 SEQUENCE
FPNWGSFDYWGQGTLVTVSSASIKG

SEQ ID NO: 37 - protein, 9 aa, Artificial Sequence
  CDR H3 SEQUENCE
VPNWGSLDA

SEQ ID NO: 38 - protein, 9 aa, Artificial Sequence
  CDR H3 SEQUENCE
FPNWGSFDY

SEQ ID NO: 39 - protein, 5 aa, Artificial Sequence
  CDL3
  MISC_FEATURE (2)..(2): Xaa denotes Gln, Arg, Met, Ser, Thr or Val
  MISC_FEATURE (3)..(3): Xaa denotes Tyr or Val
  MISC_FEATURE (4)..(4): Xaa denotes Glu, Ala, Gly or Ser
  MISC_FEATURE (5)..(5): Xaa denotes Thr, Ala or Ser
QXXXX

SEQ ID NO: 40 - protein, 5 aa, Artificial Sequence
  CDR L3 SEQUENCE
QRYAT

SEQ ID NO: 41 - protein, 5 aa, Artificial Sequence
  CDR L3 SEQUENCE
QRYST

SEQ ID NO: 42 - protein, 5 aa, Artificial Sequence
  CDR L3 SEQUENCE
QMYST

SEQ ID NO: 43 - protein, 5 aa, Artificial Sequence
  CDR L3 SEQUENCE
QQVGT

SEQ ID NO: 44 - protein, 5 aa, Artificial Sequence
  CDR L3 SEQUENCE
QQVST

SEQ ID NO: 45 - protein, 5 aa, Artificial Sequence
  CDR L3 SEQUENCE
QQYST

SEQ ID NO: 46 - protein, 5 aa, Artificial Sequence
  CDR L3 SEQUENCE
QSYST

SEQ ID NO: 47 - protein, 5 aa, Artificial Sequence
  CDR L3 SEQUENCE
QQYAT

SEQ ID NO: 48 - protein, 5 aa, Artificial Sequence
  CDR L3 SEQUENCE
QQYSS

SEQ ID NO: 49 - protein, 5 aa, Artificial Sequence
  CDR L3 SEQUENCE
QTYST

SEQ ID NO: 50 - protein, 5 aa, Artificial Sequence
  CDR L3 SEQUENCE
QQYGS

SEQ ID NO: 51 - protein, 5 aa, Artificial Sequence
  CDR L3 SEQUENCE
QQYEA

SEQ ID NO: 52 - protein, 5 aa, Artificial Sequence
  CDR L3 SEQUENCE
QQYET

SEQ ID NO: 53 - protein, 118 aa, Artificial Sequence
  VH region
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARFPNWGSFDYWGQGTLVTVSS

SEQ ID NO: 54 - protein, 118 aa, Artificial Sequence
  VH region
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARMPNWGSFDYWGQGTLVTVSS

SEQ ID NO: 55 - protein, 118 aa, Artificial Sequence
  VH region
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCVRMPNWGSLDHWGQGTLVTVSS

SEQ ID NO: 56 - protein, 103 aa, Artificial Sequence
  VL region
EIVLTQSPGTLSLSPGERATLSCRASQSISSSYLAWYQQKPGQAPRLLIYGASSRATGIPDRFSGSGSGTDFTLTISRLEPEDFAVYYCQQYETFGQGTKVEI

SEQ ID NO: 57 - protein, 103 aa, Artificial Sequence
  VL region
EIVLTQSPGTLSLSPGERATLSCRASQSISSSYLAWYQQKPGQAPRLLIYGASSRATGIPDRFSGSGSGTDFTLTISRLEPEDFAVYYCQQYASFGQGTKVEI

SEQ ID NO: 58 - protein, 103 aa, Artificial Sequence
  VL region
EIVLTQSPGTLSLSPGERATLSCRASQSISSSYLAWYQQKPGQAPRLLIYGASSRATGIPDRFSGSGSGTDFTLTISRLEPEDFAVYYCQQYEAFGQGTKVEI

SEQ ID NO: 59 - protein, 444 aa, Artificial Sequence
  heavy chain
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARMPNWGSFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 60 - protein, 444 aa, Artificial Sequence
  heavy chain
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCVRMPNWGSLDHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 61 - protein, 444 aa, Artificial Sequence
  heavy chain
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCVRMPNWGSLDHWGQGTLVTVSSASIKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 62 - protein, 444 aa, Artificial Sequence
  heavy chain
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARMPNWGSLDHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 63 - protein, 444 aa, Artificial Sequence
  heavy chain
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARMPNWGSLDHWGQGTLVTVSSASIKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 64 - protein, 443 aa, Artificial Sequence
  heavy chain
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARMPNWGSLDHWGQGTLVTVSSASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTFRVVSVLTVLHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 65 - protein, 211 aa, Artificial Sequence
  light chain
EIVLTQSPGTLSLSPGERATLSCRASQSISSSYLAWYQQKPGQAPRLLIYGASSRATGIPDRFSGSGSGTDFTLTISRLEPEDFAVYYCQQYEAFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 66 - protein, 211 aa, Artificial Sequence
  light chain
EIVLTQSPGTLSLSPGERATLSCRASQSISSSYLAWYQQKPGQAPRLLIYGASSRATGIPDRFSGSGSGTDFTLTISRLEPEDFAVYYCQQYETFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2020/0165347 A1
(43) Pub. Date: May 28, 2020

(54) METHOD OF TREATMENT USING IL-13R ANTIBODY

(71) Applicants: Aslan Pharmaceuticals PTE LTD, Singapore (SG); CSL LIMITED, Parkville (AU)

(72) Inventors: Bertil Lindmark, Singapore (SG); Ann Gee Lisa Ooi, Singapore (SG)

(21) Appl. No.: 16/625,902

(22) PCT Filed: Jun. 29, 2018
(86) PCT No.: PCT/SG2018/050319

§ 371 Date: Dec. 23, 2019

(30) Foreign Application Priority Data
Jun. 30, 2017 (SG) 10201705435W
Apr. 30, 2018 (SG) 10201803635V

(51) Int. Cl. C07K 16/28; A61P 35/00

(52) U.S. Cl. CPC C07K 16/2866; A61K 47/6803; A61P 35/00

(57) ABSTRACT

The present disclosure provides a method of treating cutaneous T cell lymphoma (CTCL), in particular mycosis fungoides and/or Sézary syndrome, comprising administering a therapeutically effective amount of an antagonist antibody or binding fragment thereof specific to the IL-13 receptor to a patient in need thereof.

The present disclosure relates to a therapy for the treatment of cutaneous T cell lymphoma, in particular mycosis fungoides and/or Sézary syndrome.

### BACKGROUND

Cutaneous T cell lymphoma (CTCL) is a subclass of non-Hodgkin lymphoma. Unlike most non-Hodgkin lymphomas (which are generally B cell related), CTCL is caused by a mutation of T cells. The cancerous T cells in the body initially migrate to the skin, causing various lesions to appear. These lesions change shape as the disease progresses, typically beginning as what appears to be a rash, which can be very itchy, and eventually forming plaques and tumors before spreading to other parts of the body.

CTCL can be subdivided into mycosis fungoides and Sézary Syndrome. Sézary syndrome is a rare, aggressive subtype of CTCL, a distinct form, but closely related to mycosis fungoides (MF). Sézary syndrome is characterized by exfoliative erythroderma and significant numbers of circulating malignant T cells (Sézary cells). Sézary syndrome can also involve lymph nodes and visceral organs in some patients.

Mycosis fungoides, also known as Alibert-Bazin syndrome or granuloma fungoides, is the most common form of cutaneous T-cell lymphoma. It generally affects the skin, but may progress internally over time. Symptoms include rash, tumors, skin lesions, and itchy skin. While the cause remains unclear, most cases are not hereditary. Most cases are in people over 20 years of age, and it is more common in men than women.

Mycosis fungoides can be treated in a variety of ways. Common treatments include simple sunlight, ultraviolet light, topical steroids, topical and systemic chemotherapies, local superficial radiotherapy, the histone deacetylase inhibitor vorinostat, total skin electron beam radiation, photopheresis and systemic therapies (e.g. interferons, retinoids, rexinoids) or biological therapies. Treatments are often used in combination.

If treatment is successful, the disease can go into remission and remission can last indefinitely. Treatments may halt disease progression, and this is called stable disease. Stable disease may also last indefinitely but is a less satisfactory situation.

Unfortunately, even with treatment the disease may progress, to involve nodes, blood and internal organs, or transform into a higher-grade lymphoma. The disease is incurable, but many patients experience prolonged periods of disease-control. Quality of life and maximizing periods of remission or stable disease, while minimizing treatments and toxicities, are two primary objectives in clinical care.

In 2010, the U.S. Food and Drug Administration granted orphan drug designation for naloxone lotion, a topical opioid receptor competitive antagonist used as a treatment for pruritus in cutaneous T-cell lymphoma.

There is still a need for alternative treatments for cutaneous T cell lymphoma, such as mycosis fungoides and Sézary Syndrome. The present inventors have data, which suggests that an antagonist antibody or binding fragment thereof specific to IL-13 receptor will be a useful therapy.

### SUMMARY OF THE DISCLOSURE

The present disclosure will be summarized in paragraphs below. Thus, there is provided:

1. A method of treating cutaneous T cell lymphoma comprising administering a therapeutically effective amount of an antagonist antibody or a binding fragment thereof specific to the IL-13 receptor to a patient in need thereof.

2. A method according to paragraph 1 wherein the antibody or binding fragment thereof is specific to IL-13R alpha 1 (IL-13Rα1) or anti-IL-13R alpha 2 (IL-13Rα2).

3. The method according to paragraph 2, wherein the antibody or binding fragment thereof is an anti-IL-13Rα1 antibody.

4. The method according to paragraph 2 or 3, wherein the antibody or binding fragment thereof binds the epitope FFYQ.

5. The method according to any one of paragraph 1 to 4, wherein the antibody or binding fragment thereof has a heavy chain variable domain comprising a CDR H1, CDR H2 and a CDR H3 with a sequence shown in SEQ ID NO: 1, 2 and 3 or 4 (or any one of sequences 12 to 38) respectively or a variable domain wherein one, two or three amino acids in the CDRs are added, substituted or deleted.

6. The method according to any one of paragraphs 1 to 5, wherein the antibody or binding fragment thereof has a light chain variable domain comprising a CDR L1, CDR L2 and a CDR L3 with a sequence shown in SEQ ID NO: 5, 6 and 7 (or any one of sequences 39 to 52) respectively or a variable domain wherein one, two or three amino acids in the CDRs are added substituted or deleted.

7. The method according to any one of paragraphs 1 to 6, wherein the antibody or binding fragment thereof has a light chain variable domain comprising a sequence shown in SEQ ID NO: 9 or a sequence as least 95% percent identical thereto (which retains specificity for IL-13 receptor, in particular IL-13Rα1).

8. The method according to any one of paragraphs 1 to 6, wherein the antibody or binding fragment thereof has a light chain variable domain comprising a sequence of SEQ ID NO: 9.

9. The method according to any one of paragraphs 1 to 8, wherein the antibody or binding fragment thereof has a heavy chain variable domain comprising a sequence shown in SEQ ID NO: 8 or a sequence at least 95% percent identical thereto (which retains specificity for IL-13 receptor, in particular IL-13Rα1).

10. The method according to any one of paragraphs 1 to 9, wherein the antibody or binding fragment thereof has a heavy chain variable domain comprising a sequence shown in SEQ ID NO: 8.

11. The method according to any one of paragraphs 1 to 10, wherein the antibody or binding fragment thereof is human or humanized.

12. The method according to any one of paragraphs 1 to 11, wherein the antibody binding fragment is selected from the group consisting of an Fv, dsFv, scFv, Fab, Fab' or F(ab')₂ fragment.

13. The method according to any one of paragraphs 1 to 11, wherein the antibody is a full-length antibody.

14. The method according to paragraph 13, wherein the antibody heavy chain has the sequence shown in SEQ ID NO: 10 or a sequence at least 95% identical thereto, in particular SEQ ID NO: 10.

15. The method according to paragraph 13 or 14, wherein the antibody light chain has the sequence shown in SEQ ID NO: 11 or a sequence at least 95% identical thereto, in particular SEQ ID NO: 11.

16. The method according to any one of paragraphs 1 to 15, wherein the antibody or binding fragment thereof inhibits IL-13 signaling through the IL-13 receptor complex.

17. The method according to any one of paragraphs 1 to 16, wherein the CTCL is refractory, for example to first line treatment.

18. A method according to any one of paragraphs 1 to 17, wherein the lymphoma is mycosis fungoides.

19. The method according to any one of paragraphs 1 to 18, wherein the lymphoma is Sézary syndrome.

20. The method according to any one of paragraphs 1 to 19, wherein the antibody or binding fragment thereof is administered as a pharmaceutical formulation, for example a parenteral formulation.

21. The method according to any one of paragraphs 1 to 20, wherein the anti-IL-13 receptor antibody is administered at a dose in the range of 1 ng to 1000 μg.

22. The method according to any one of paragraphs 1 to 21, wherein the antibody or antibody binding fragment is employed as a monotherapy.

23. The method according to any one of paragraphs 1 to 21, where the antibody or binding fragment is employed as part of a combination therapy comprising a further therapeutic agent.

24. The method according to paragraph 23, wherein the further therapeutic agent is an anti-cancer agent, for example a chemotherapeutic agent or combination of chemotherapeutic agents, such as selected from the group comprising of a platin (such as cisplatin or oxaliplatin), gemcitabine, capecitabine, 5-FU, FOLFOX, FOLFIRI, FLOFIRINOX and a combination of two or more of the same.

25. The method according to paragraph 23 or 24, wherein the therapeutic agent is an immune modifying agent such as a cytokine, for example selected from the group comprising: IL-13, IL-12 and IL-2.

26. The method according to any one of paragraphs 1 to 25, wherein the antibody is conjugated to a payload.

27. The method according to paragraph 26, wherein the payload is a toxin or a drug.

28. An antagonist antibody or binding fragment thereof specific to the IL-13 receptor for use in treating cutaneous T cell lymphoma.

29. An antagonist antibody or binding fragment for use according to paragraph 28, wherein the antibody or binding fragment thereof is specific to IL-13R alpha 1 (IL-13Rα1) or anti-IL-13R alpha 2 (IL-13Rα2).

30. An antagonist antibody or binding fragment for use according to paragraph 29, wherein the antibody is an anti-IL-13Rα1 antibody.

31. An antagonist antibody or binding fragment for use according to paragraph 29 or 30, wherein the antibody binds the epitope FFYQ.

32. An antagonist antibody or binding fragment for use according to any one of paragraphs 28 to 31, wherein the antibody has a heavy chain variable domain comprising a CDR H1, CDR H2 and a CDR H3 with a sequence shown in SEQ ID NO: 1, 2, 3 or 4 (or any one of sequences 12 to 38) respectively or a variable domain wherein one, two or three amino acids in the CDRs are independently added substituted or deleted.

33. An antagonist antibody or binding fragment for use according to any one of paragraph 28 to 32, wherein the antibody has a light chain variable domain comprising a CDR L1, CDR L2 and a CDR L3 with a sequence shown in SEQ ID NO: 5, 6 and 7 (or any one of sequences 39 to 52) respectively or a variable domain wherein one, two or three amino acids in the CDRs are independently added substituted or deleted.

34. An antagonist antibody or binding fragment for use according to any one of paragraphs 28 to 33, wherein the antibody has a light chain variable domain comprising a sequence shown in SEQ ID NO: 9 or a sequence at least 95% percent identical thereto (which retains specificity for IL-13 receptor, in particular IL-13 Rα1).

35. An antagonist antibody or binding fragment for use according to any one of paragraphs 28 to 34, wherein the antibody has a light chain variable domain comprising a sequence shown in SEQ ID NO: 9.

36. An antagonist antibody or binding fragment for use according to any one of paragraphs 28 to 35, wherein the antibody has a heavy chain variable domain comprising a sequence shown in SEQ ID NO: 8 or a sequence at least 95% percent identical thereto (which retains specificity for IL-13 receptor, in particular IL-13 Rα1).

37. An antagonist antibody or binding fragment for use according to any one of paragraphs 28 to 36, wherein the antibody has a heavy chain variable domain comprising a sequence shown in SEQ ID NO: 8.

38. An antagonist antibody or binding fragment for use according to any one of paragraphs 28 to 37, wherein the antibody is human or humanized.

39. An antagonist antibody or binding fragment for use according to any one of paragraphs 28 to 38, wherein the antibody binding fragment is selected from the group comprising an Fv, dsFv, scFv, Fab, Fab' or F(ab')₂ fragment.

40. An antagonist antibody or binding fragment for use according to any one of paragraphs 28 to 38, wherein he antibody is a full-length antibody.

41. An antagonist antibody or binding fragment for use according to paragraph 40, wherein the antibody heavy chain has the sequence shown in SEQ ID NO: 10 or a sequence at least 95% identical thereto, such as SEQ ID NO: 10.

42. An antagonist antibody or binding fragment for use according to paragraph 40 or 41, wherein the antibody light chain has the sequence shown in SEQ ID NO: 11 or a sequence at least 95% identical thereto, such as SEQ ID NO: 11.

43. An antagonist antibody or binding fragment for use according to any one of paragraphs 28 to 42, wherein the antibody or binding fragment thereof inhibits IL-13 signaling through the IL-13 receptor complex.

44. An antagonist antibody or binding fragment for use according to any one of paragraphs 28 to 43, wherein the CTCL is refractory, for example to first line treatment.

45. An antagonist antibody or binding fragment for use according to any one of paragraphs 28 to 44, wherein the lymphoma is mycosis fungoides.

46. An antagonist antibody or binding fragment for use according to any one of paragraphs 28 to 45, wherein the lymphoma is Sézary syndrome.

47. An antagonist antibody or binding fragment for use according to any one of paragraphs 28 to 46, wherein the antibody or binding fragment is administered as a pharmaceutical formulation, for example a parenteral formulation.

48. An antagonist antibody or binding fragment for use according to any one of paragraphs 28 to 47, wherein the anti-IL-13 receptor antibody or binding fragment is administered at a dose in the range of 1 ng to 1000 μg.

49. An antagonist antibody or binding fragment for use according to any one of paragraphs 28 to 48, wherein the antibody or antibody binding fragment is employed as a monotherapy.

50. An antagonist antibody or binding fragment for use according to any one of paragraphs 28 to 48, where the antibody or binding fragment is employed as part of a combination therapy comprising a further therapeutic agent.

51. An antagonist antibody or binding fragment for use according to paragraph 50, wherein the further therapeutic agent is an anti-cancer agent, for example a chemotherapeutic agent or combination of chemotherapeutic agents, such as selected from the group comprising of a platin (such as cisplatin or oxaliplatin), gemcitabine, capecitabine, 5-FU, FOLFOX, FOLFIRI, FLOFIRINOX and a combination of two or more of the same.

52. An antagonist antibody or binding fragment for use according to paragraph 50 or 51, wherein the therapeutic agent is an immune modifying agent such as a cytokine, for example selected from the group comprising: IL-13, IL-12 and IL-2.

53. An antagonist antibody or binding fragment for use according to any one of paragraphs 28 to 52, wherein the antibody or binding fragment is conjugated to payload.

54. An antagonist antibody or binding fragment for use according to paragraph 53, wherein the payload is a toxin or a drug.

55. Use of an antagonist antibody or binding fragment thereof specific to the IL-13 receptor for the manufacture of a medicament for treating cutaneous T cell lymphoma.

56. Use of an antagonist antibody or binding fragment according to paragraph 55, wherein the antibody or binding fragment thereof is specific to IL-13R alpha 1 (IL-13Rα1) or anti-IL-13R alpha 2 (IL-13Rα2).

57. Use of an antagonist antibody or binding fragment according to paragraph 56, wherein the antibody is an anti-IL-13Rα1 antibody.

58. Use of an antagonist antibody or binding fragment according to paragraph 56 or 57, wherein the antibody binds the epitope FFYQ.

59. Use of an antagonist antibody or binding fragment according to any one of paragraphs 55 to 58, wherein the antibody or binding fragment has a heavy chain variable domain comprising a CDR H1, CDR H2 and a CDR H3 with a sequence shown in SEQ ID NO: 1, 2 and 3 or 4 (or any one of sequences 12 to 38) respectively or a variable domain where one, two or three amino acids in the CDRs are independently added, deleted or substituted.

60. Use of an antagonist antibody or binding fragment according to any one of paragraphs 55 to 59, wherein the antibody has a heavy chain variable domain comprising a CDR L1, CDR L2 and a CDR L3 with a sequence shown in SEQ ID NO: 5, 6 and 7 (or any one of sequence 39 to 52) respectively or a sequence where one, two or three amino acids in the CDRs are independently added, deleted or substituted.

61. Use of an antagonist antibody or binding fragment according to any one of paragraphs 55 to 60, wherein the antibody has a light chain variable domain comprising a sequence shown in SEQ ID NO: 9 or a sequence at least 95% percent identical thereto (which retains specificity for IL-13 receptor, in particular IL-13 Rα1).

62. Use of an antagonist antibody or binding fragment according to any one of paragraphs 55 to 61, wherein the antibody has a light chain variable domain comprising a sequence shown in SEQ ID NO: 9.

63. Use of an antagonist antibody or binding fragment according to any one of paragraphs 55 to 62, wherein the antibody has a heavy chain variable domain comprising a sequence shown in SEQ ID NO: 8 or a sequence at least 95% percent identical thereto (which retains specificity for IL-13 receptor, in particular IL-13 Rα1).

64. Use of an antagonist antibody or binding fragment according to any one of paragraphs 55 to 63, wherein the antibody has a heavy chain variable domain comprising a sequence shown in SEQ ID NO: 8.

65. Use of an antagonist antibody or binding fragment according to any one of paragraphs 55 to 64, wherein the antibody is human or humanized.

66. Use of an antagonist antibody or binding fragment according to any one of paragraphs 55 to 65, wherein the antibody binding fragment is selected from the group comprises an Fv, dsFv, scFv, Fab, Fab' or F(ab')₂ fragment.

67. Use of an antagonist antibody or binding fragment according to any one of paragraphs 55 to 65, wherein the antibody is a full-length antibody.

68. Use of an antagonist antibody or binding fragment according to paragraph 67, wherein the antibody heavy chain has the sequence shown in SEQ ID NO: 10 or a sequence at least 95% identical thereof, such as SEQ ID NO: 10.

69. Use of an antagonist antibody or binding fragment according to paragraph 67 or 68, wherein the antibody light chain has the sequence shown in SEQ ID NO: 11 or a sequence at least 95% identical thereto, such as SEQ ID NO: 11.

70. Use of an antagonist antibody or binding fragment according to any one of paragraphs 55 to 69, wherein the antibody or binding fragment thereof inhibits IL-13 signaling through the IL-13 receptor complex.

71. Use of an antagonist antibody or binding fragment according to any one of paragraphs 55 to 70, wherein the CTCL is refractory, for example to first line treatment.

72. Use of an antagonist antibody or binding fragment according to any one of paragraphs 55 to 71, wherein the lymphoma is mycosis fungoides.

73. Use of an antagonist antibody or binding fragment according to any one of paragraphs 55 to 72, wherein the lymphoma is Sézary syndrome.

74. Use of an antagonist antibody or binding fragment according to any one of paragraphs 55 to 73, wherein the antibody or binding fragment thereof is administered as a pharmaceutical formulation, for example a parenteral formulation.

75. Use of an antagonist antibody or binding fragment according to any one of paragraphs 55 to 74, wherein the anti-IL-13 receptor antibody or binding fragment thereof is administered at a dose in the range of 1 ng to 1000 μg.

76. Use of an antagonist antibody or binding fragment according to any one of paragraphs 55 to 75, wherein the antibody or antibody binding fragment is employed as a monotherapy.

77. Use of an antagonist antibody or binding fragment according to any one of paragraphs 55 to 76, where the antibody or binding fragment is employed as part of a combination therapy comprising a further therapeutic agent

78. Use of an antagonist antibody or binding fragment according to paragraph 77, wherein the further therapeutic agent is an anti-cancer agent, for example a chemotherapeutic agent or combination of chemotherapeutic agents, such as selected from the group comprising of a platin (such as cisplatin or oxaliplatin), gemcitabine, capecitabine, 5-FU, FOLFOX, FOLFIRI, FLOFIRINOX and a combination of two or more of the same.

79. Use of an antagonist antibody or binding fragment according to paragraph 77 or 78, wherein the therapeutic agent is an immune modifying agent such as a cytokine, for example selected from the group comprising: IL-13, IL-12 and IL-2.

80. Use of an antagonist antibody or binding fragment according to any one of paragraphs 55 to 79, wherein the antibody or binding fragment is conjugated to a payload.

81. Use of an antagonist antibody or binding fragment according to paragraph 80, wherein the payload is a toxin or a drug.

In one embodiment the IL13-R1α1 antibody or binding fragment employed in the f the present disclosure comprises a CDRH3 independently selected from a sequence comprising SEQ ID NO: 13 to 38.

In one embodiment, the anti-IL13R antibody or binding fragment employed in the present disclosure comprises a VH CDR1 comprising an amino acid sequence as set forth in SEQ ID NO: 1, a VH CDR2 comprising an amino acid sequence as set forth in SEQ ID NO: 2, and a VH CDR3 comprising an amino acid sequence as set forth in SEQ ID NO: 3 or 12.

In one embodiment, the anti-IL13R antibody or binding fragment employed in the present disclosure comprises a CDRH1 comprising an amino acid sequence as set forth in SEQ ID NO: 1, a CDRH2 comprising an amino acid sequence as set forth in SEQ ID NO: 2, and a CDRH3 comprising an amino acid sequence as set forth in SEQ ID NO: 4, 13, 14, 15, 16, 17, 18, 19, 20, 21, 22, 23, 24, 25, 26, 27, 28, 29, 30, 31, 32, 33, 34, 35, 36, 37 or 38.

In one embodiment, the anti-IL13R antibody or binding fragment employed in the present disclosure comprises a CDRH1 comprising an amino acid sequence as set forth in SEQ ID NO: 1, a CDRH2 comprising an amino acid sequence as set forth in SEQ ID NO: 2, and a CDRH3 comprising an amino acid sequence as set forth in SEQ ID NO: 4.

In one embodiment CDRL1 is an amino acid sequence comprising SEQ ID NO: 5.

In one embodiment CDRL2 is an amino acid sequence comprising SEQ ID NO: 6.

In one embodiment CDL3 comprises SEQ ID NO: 39

In one embodiment the IL-13Rα1 antibody employed in the formulation of the present disclosure comprises a CDRL3 independently selected from a sequence comprising SEQ ID NO: 40 to 52:

In one embodiment, the anti-IL-13Rα antibody or binding fragment employed in the present disclosure comprises a CDRL1 comprising an amino acid sequence SEQ ID NO: 5, a CDRL2 comprising an amino acid sequence SEQ ID NO: 6, and a CDRL3 comprising an amino acid sequence as set forth in SEQ ID NO:39.

In one embodiment, the anti-IL-13Rα antibody of the present disclosure comprises a VL CDRL1 comprising an amino acid sequence SEQ ID NO: 5, CDRL2 comprising an amino acid sequence SEQ ID NO: 6, and a CDRL3 comprising an amino acid sequence as set forth in SEQ ID NO: 7, 40, 41, 42, 43, 44, 45, 46, 47, 48, 49, 50, 51 or 52.

In one embodiment, the anti-IL-13Rα antibody of the present disclosure comprises a CDRL1 comprising an amino acid sequence SEQ ID NO: 31, a CDRL2 comprising an amino acid sequence SEQ ID NO: 6, and a CDRL3 comprising an amino acid sequence as set forth in SEQ ID NO: 7.

In one embodiment, the anti-IL13R antibody of the present disclosure comprises a CDRH1 comprising an amino acid sequence as set forth in SEQ ID NO: 1, a CDRH2 comprising an amino acid sequence as set forth in SEQ ID NO: 2, and a CDRH3 comprising an amino acid sequence as set forth in SEQ ID NO: or 3 or 12, a CDRL1 comprising an amino acid sequence SEQ ID NO: 5, a CDRL2 comprising an amino acid sequence SEQ ID NO: 6, and a CDRL3 comprising an amino acid sequence as set forth in SEQ ID NO: 39.

In one embodiment, the anti-IL13R antibody of the present disclosure comprises a CDRH1 comprising an amino acid sequence as set forth in SEQ ID NO: 1, a CDRH2 comprising an amino acid sequence as set forth in SEQ ID NO: 2, and a CDRH3 comprising an amino acid sequence as set forth in SEQ ID NO: 3, 4 or 12, a CDRL1 comprising an amino acid sequence SEQ ID NO: 5, a CDRL2 comprising an amino acid sequence SEQ ID NO: 6, and a CDRL3 comprising an amino acid sequence as set forth in SEQ ID NO: 7, 40, 41, 42, 43, 44, 45, 46, 47, 48, 49, 50, 51 or 52.

In one embodiment, the anti-IL13R antibody of the present disclosure comprises a CDRH1 comprising an amino acid sequence as set forth in SEQ ID NO: 1, a CDRH2 comprising an amino acid sequence as set forth in SEQ ID NO: 2, and a CDRH3 comprising an amino acid sequence as set forth in SEQ ID NO: 3, 4 or 12, a CDRL1 comprising an amino acid sequence SEQ ID NO: 5, a CDRL2 comprising an amino acid sequence SEQ ID NO: 6, and a CDRL3 comprising an amino acid sequence as set forth in SEQ ID NO: 7.

In one embodiment, the anti-IL13R antibody of the present disclosure comprises a CDRH1 comprising an amino acid sequence as set forth in SEQ ID NO: 1, a CDRH2 comprising an amino acid sequence as set forth in SEQ ID NO: 2, and a CDRH3 comprising an amino acid sequence as set forth in SEQ ID NO: 4, a CDRL1 comprising an amino acid sequence SEQ ID NO: 5, a CDRL2 comprising an amino acid sequence SEQ ID NO: 6, and a CDRL3 comprising an amino acid sequence as set forth in SEQ ID NO: 7.

In one embodiment the VH region is independently selected from a sequence comprising SEQ ID NO: 8, 53, 54 or 55.

In one embodiment the VL is independently selected from a sequence comprising SEQ ID NO: 56, 57 or 58.

In one embodiment the VH sequence is SEQ ID NO: 53 (or a sequence at least 95% identical thereto) and the VL sequence is SEQ ID NO: 56, SEQ ID NO: 9 or 57, SEQ ID NO: 58 or SEQ ID NO: 55 (or a sequence at least 95% identical to any one of the same).

In one embodiment the VH sequence is SEQ ID NO: 54 (or a sequence at least 95% identical thereto) and the VL sequence is SEQ ID NO: 9; SEQ ID NO: 56, SEQ ID NO: 57, or SEQ ID NO: 58 (or a sequence at least 95% identical to any one of the same).

In one embodiment the VH sequence is SEQ ID NO: 55 (or a sequence at least 95% identical thereto) and the VL sequence is SEQ ID NO: 9; SEQ ID NO: 56, SEQ ID NO: 57, or SEQ ID NO: 58 (or a sequence at least 95% identical to any one of the same).

In one embodiment the VH sequence is SEQ ID NO: 8 (or a sequence at least 95% identical thereto) and the VL sequence is SEQ ID NO: 9, SEQ ID NO: 56, SEQ ID NO: 57, or SEQ ID NO: 58 (or a sequence at least 95% identical to any one of the same).

In one embodiment the VL sequence is SEQ ID NO: 56 (or a sequence at least 95% identical thereto) and the VH sequence is SEQ ID NO: 53, SEQ ID NO: 54, SEQ ID NO: 55 or SEQ ID NO: 8. (or a sequence at least 95% identical to any one of the same)

In one embodiment the VL sequence is SEQ ID NO: 9 or 57 (or a sequence at least 95% identical thereto) and the VH sequence is SEQ ID NO: 53, SEQ ID NO: 54, SEQ ID NO: 55 or SEQ ID NO: 8 (or a sequence at least 95% identical to any one of the same).

In one embodiment the VL sequence is SEQ ID NO: 58 (or a sequence at least 95% identical thereto) and the VH sequence is SEQ ID NO: 53, SEQ ID NO: 54, SEQ ID NO: 55 or SEQ ID NO: 8 (or a sequence at least 95% identical to any one of the same). In one embodiment the VH sequence is SEQ ID NO: 8 (or a sequence at least 95% identical thereto) and the VL sequence is SEQ ID NO: 9 or 57((or a sequence at least 95% identical thereto).

Variable region as employed herein refers to the region in an antibody chain comprising the CDRs and a suitable framework.

In one embodiment the heavy chain comprises a sequence independently selected from: SEQ ID NO: 10, 59, 60, 61, 62, 63, 64 or a sequence at least 95% identical to any one of the same.

In one embodiment the light chain is independently selected from SEQ ID NO: 11, SEQ ID NO: 65, SEQ ID NO: 66 or a sequence at least 95% identical to any one of the same.

In one embodiment the heavy chain is independently selected from SEQ ID NO: 10, 59, 60, 61, 62, 63 and 64 (or a sequence at least 95% identical to any one of the same) and the light chain is independently selected from SEQ ID NO: 11, 65 and 66 (or a sequence at least 95% identical to any one of the same).

In one embodiment the heavy chain is SEQ ID NO: 10 (or a sequence at least 95% identical thereto) and the light chain is independently selected from SEQ ID NO: 11, 65 and 66 (or a sequence at least 95% identical to any one of the same).

In one embodiment the heavy chain is SEQ ID NO: 59 (or a sequence at least 95% identical thereto) and the light chain is independently selected from SEQ ID NO: 11, 65 and 66 (or a sequence at least 95% identical to any one of the same).

In one embodiment the heavy chain is SEQ ID NO: 60 (or a sequence at least 95% identical thereto) and the light chain is independently selected from SEQ ID NO: 11, 65 and 66 (or a sequence at least 95% identical to any one of the same).

In one embodiment the heavy chain is SEQ ID NO: 61 (or a sequence at least 95% identical thereto) and the light chain is independently selected from SEQ ID NO: 11, 65 and 66 (or a sequence at least 95% identical to any one of the same).

In one embodiment the heavy chain is SEQ ID NO: 62 (or a sequence at least 95% identical thereto) and the light chain is independently selected from SEQ ID NO: 11, 65 and 66 (or a sequence at least 95% identical to any one of the same). In one embodiment the heavy chain is SEQ ID NO: 63 (or a sequence at least 95% identical thereto) and the light chain is independently selected from SEQ ID NO: 11, 65 and 66 (or a sequence at least 95% identical to any one of the same). In one embodiment the heavy chain is SEQ ID NO: 64 (or a sequence at least 95% identical thereto) and the light chain is independently selected from SEQ ID NO: 11, 65 and 66 (or a sequence at least 95% identical to any one of the same).

In one embodiment the heavy chain is SEQ ID NO: 62 or 64 (or a sequence at least 95% identical to any one of the same) and a light chain with the sequence shown in SEQ ID NO: 11 (or a sequence at least 95% identical thereto).

In one embodiment the heavy chain is SEQ ID NO: 62 (or a sequence at least 95% identical to any one of the same) and a light chain with the sequence shown in SEQ ID NO: 11 (or a sequence at least 95% identical thereto).

In one embodiment the heavy chain is SEQ ID NO: 64 (or a sequence at least 95% identical to any one of the same) and a light chain with the sequence shown in SEQ ID NO: 11 (or a sequence at least 95% identical thereto).

In one embodiment the heavy chain is SEQ ID NO: 10 (or a sequence at least 95% identical to any one of the same) and a light chain with the sequence shown in SEQ ID NO: 11 (or a sequence at least 95% identical thereto).

### SUMMARY OF THE FIGURES

FIG. 1 ASLAN004 anti-tumor activity in a sample from a patient with primary CTCL.

FIG. 2 Shows expression of IL-13Rα1 as determined by flow cytometry in Hut-78 cells

FIG. 3 Shows ASLAN004 Anti-tumour activity in HUT-78 cells

### SEQUENCES

Sequences 1, 2, 4 to 11 and 13 to 66 are only contained in the sequence listing

### SEQ ID NO: 3 CDRH3

Met Pro Asn Trp Gly Ser Xaa Asp Xbb

Xaa is: Phe or Leu

Xbb is: His, Tyr, Thr, or Ser

### SEQ ID NO: 12 CDRH3

X₁ Pro Asn Trp Gly X₆ X₇ Asp X₉

X1 denotes Phe, Met, Gln, Leu or Val

X6 denotes Ser or Ala

X7 denotes Phe, Leu, Ala or Met

X9 denotes Tyr, Gln, Lys, Arg, Trp, His, Ala, Thr, Ser, Asn or Gly

SEQ ID NO: 39 CDRL3 Gln X₂X₃X₄X₅

X2 denotes Gln, Arg, Met, Ser, Thr or Val.

X3 denotes Tyr or Val.

X4 denotes Glu, Ala, Gly or Ser.

X5 denotes Thr, Ala or Ser.

### DETAILED DISCLOSURE

Cutaneous T cell lymphoma as employed herein refers to a rare type of non-Hodgkin lymphoma that affects the skin. It is caused by white blood cells, called T-cell lymphocytes, growing in an uncontrolled way, for example causing raised, rash-like or itchy patches of skin, lumps on the skin and/or swollen lymph nodes. As mentioned above it can be divided into mycosis fungoides, Sézary Syndrome, CD 30+ cutaneous T-cell lymphoma and extranodal NK/T-cell lymphoma (nasal type).

Sézary Syndrome is a rare and aggressive type of cutaneous T cell lymphoma, which is closely related to mycosis fungoides. As mentioned above patients have exfoliative erythroderma and significant numbers of circulating malignant T cells (Sézary cells). The condition can also involve the lymph nodes and visceral organs.

Sézary Syndrome can develop from mycosis fungoides or the full symptoms may develop de novo.

Mycosis fungoides, also known as Alibert-Bazin syndrome or granuloma fungoides, is the most common form of cutaneous T-cell lymphoma. It generally affects the skin but may progress internally over time. Symptoms include rash, tumors, skin lesions, and itchy skin. Pagetoid reticulosis (also known as “acral mycoses fungoides”, “localized epidermotropic reticulosis”, “mycosis fungoides palmaris et plantaris”, “unilesional mycosis fungoides”, and “Woringer-Kolopp disease”) can be considered to be a form of mycosis fungoides.

The disease is an unusual expression of CD4 T cells. These T cells are skin-associated, meaning that they biochemically and biologically are most related to the skin, in a dynamic manner. Diagnosis is sometimes difficult because the early phases of the disease often resemble eczema or even psoriasis. Diagnosis is generally accomplished through a skin biopsy. Several biopsies are recommended, to be more certain of the diagnosis. The diagnosis is made through a combination of the clinical picture and examination and is confirmed by biopsy.

Premycotic is a phase of mycosis fungoides in which a patient has areas of red, scaly, itchy skin on areas of the body that are not usually exposed to sun. This early-phase mycosis fungoides is hard to diagnose. The premycotic phase may last from months to decades.

Secondary cutaneous CD30+ large-cell lymphoma is a cutaneous condition that may arise in cases of mycosis fungoides, and in patients with lymphomatoid papulosis.

To stage the mycosis fungoides, various tests may be employed to assess nodes, blood and internal organs, but most patients present with disease apparently confined to the skin, as patches (flat spots) and plaques (slightly raised or ‘wrinkled’ spots).

Extranodal NK/T-cell lymphoma, nasal type, which was known as angiocentric lymphoma in the REAL classification, and also as nasal-type NK lymphoma, NK/T-cell lymphoma, and polymorphic/malignant midline reticulosis is a cutaneous condition which in Korea is reported to be the most common form of cutaneous lymphoma after mycosis fungoides.

### Staging for Mycosis Fungoides and Sezary Syndrome

Mycosis fungoides (MF) and Sezary syndrome (SS) are staged based on 4 factors:

T describes how much of the skin is affected by the lymphoma (tumor).

N describes the extent of the lymphoma in the lymph nodes.

M is for the spread (metastasis) of the lymphoma to other organs.

B is for lymphoma cells in the blood.

### T Categories

T1: Skin lesions can be small patches (flat lesions), papules (small bumps), and/or plaques (raised or lowered, flat lesions), but the lesions cover less than 10% of the skin surface.

T2: The patches, papules, and/or plaques cover 10% or more of the skin surface.

T3: At least one of the skin lesions is a tumor that is at least 1 centimeter (cm) across (a cm is a little less than ½ inch).

T4: The skin lesions have spread, grown larger, and grown together to cover at least 80% of the skin surface.

### N Categories

N0: Lymph nodes are not enlarged and a lymph node biopsy is not needed.

N1: Lymph nodes are enlarged, but the patterns of cells look normal or close to normal under the microscope.

N2: Lymph nodes are enlarged, and the patterns of cells look more abnormal under the microscope.

N3: Lymph nodes are enlarged, and the patterns of cells look very abnormal under the microscope.

NX: Lymph nodes are enlarged but haven't been removed (biopsied) to be looked at under the microscope.

### M Categories

M0: The lymphoma cells have not spread outside the skin or lymph nodes.

M1: Lymphoma cells have spread to other organs or tissues, such as the liver or spleen.

### B Categories

B0: Less than 5% of lymphocytes in the blood are Sezary (lymphoma) cells.

B1: Low numbers of Sezary cells in the blood (more than in B0 but less than in B2).

B2: High number of Sezary cells in the blood.

### Stage Grouping

Once the values for T, N, M, and B are known, they are combined to determine the overall stage of the lymphoma. This process is called stage grouping.

### Stage IA: T1, N0, M0, B0 or B1.

There are skin lesions but no tumors. Skin lesions cover less than 10% of the skin surface (T1), the lymph nodes are not enlarged (N0), lymphoma cells have not spread to other organs or tissues (M0), and the number of Sezary cells in the blood is not high (B0 or B1).

### Stage IB: T2, N0, M0, B0 or B1.

There are skin lesions but no tumors. Skin lesions cover at least 10% of the skin surface (T2), the lymph nodes are not enlarged (N0), lymphoma cells have not spread to other organs or tissues (M0), and the number of Sezary cells in the blood is not high (B0 or B1).

### Stage IIA: T1 or T2, N1 or N2, M0, B0 or B1.

There are skin lesions but no tumors. Skin lesions can cover up to 80% of the skin surface (T1 or T2). Lymph nodes are enlarged but the patterns of cells do not look very abnormal under the microscope (N1 or N2). Lymphoma cells have not spread to other organs or tissues (M0), and the number of Sezary cells in the blood is not high (B0 or B1).

### Stage IIB: T3, N0 to N2, M0, B0 or B1.

At least one of the skin lesions is a tumor that is 1 cm across or larger (T3). The lymph nodes are either normal (N0) or are enlarged but the patterns of cells do not look very abnormal under the microscope (N1 or N2). Lymphoma cells have not spread to other organs or tissues (M0), and the number of Sezary cells in the blood is not high (B0 or B1).

### Stage IIIA: T4, N0 to N2, M0, B0.

Skin lesions cover at least 80% of the skin surface (T4). The lymph nodes are either normal (N0) or are enlarged but the patterns of cells do not look very abnormal under the microscope (N1 or N2). Lymphoma cells have not spread to other organs or tissues (M0), and there are few (or no) Sezary cells in the blood (B0).

### Stage IIIB: T4, N0 to N2, M0, B1

Skin lesions cover at least 80% of the skin surface (T4). The lymph nodes are either normal (N0) or are enlarged but the patterns of cells do not look very abnormal under the microscope (N1 or N2). Lymphoma cells have not spread to other organs or tissues (M0), and the number of Sezary cells in the blood is low (B1).

### Stage IVA1: any T, N0 to N2, M0, B2.

Skin lesions can cover any amount of the skin surface (any T). The lymph nodes are either normal (N0) or are enlarged, but the patterns of cells do not look very abnormal under the microscope (N1 or N2). Lymphoma cells have not spread to other organs or tissues (M0), and the number of Sezary cells in the blood is high (B2).

### Stage IVA2: any T, N3, M0, any B.

Skin lesions can cover any amount of the skin surface (any T). Some lymph nodes are enlarged and the patterns of cells look very abnormal under the microscope (N3). Lymphoma cells have not spread to other organs or tissues (M0). Sezary cells may or may not be in the blood (any B).

### Stage IVB: any T, any N, M1, any B

Skin lesions can cover any amount of the skin surface (any T). The lymph nodes may be normal or abnormal (any N), and Sezary cells may or may not be in the blood (any B). Lymphoma cells have spread to other organs or tissues, such as the liver or spleen (M1).

Staging for other Skin Lymphomas

The staging system for types of skin lymphoma other than mycosis fungoides and Sezary syndrome is still fairly new, and doctors are still trying to determine how useful it is. The system is based on 3 factors:
T describes how much of the skin is affected by the lymphoma (tumor).
N describes the extent of the lymphoma in the lymph nodes.
M is for the spread (metastasis) of the lymphoma to other organs.
   For these lymphomas, only the T category is used at the time of diagnosis. If sites besides the skin (such as lymph nodes) are involved at the time of diagnosis, these lymphomas are no longer considered skin lymphomas and are staged like regular non-Hodgkin lymphoma. The N and M categories are only used if the lymphoma progresses (continues to grow) during treatment or comes back after treatment.

### T Categories

T1: There is only a single skin lesion.

T1a: The skin lesion is less than 5 cm (about 2 inches) across.

T1b: The skin lesion is larger than 5 cm across.

T2: There are 2 or more lesions on the skin. These may be in a single body region or in 2 body regions that are next to each other.

T2a: All of the skin lesions could be placed within a circle that is 15 cm (about 6 inches) across.

T2b: The circle needed to surround all of the skin lesions is larger than 15 cm across, but smaller than 30 cm (about 1 foot) across.

T2c: The circle needed to surround all of the skin lesions is larger than 30 cm across.

T3: There are skin lesions in body regions that aren't next to each other, or in at least 3 different body regions.

T3a: There are many lesions involving 2 body regions that aren't next to each other.

T3b: There are many lesions involving 3 or more body regions.

### N Categories

N0: No lymph node is enlarged or contains lymphoma cells.

N1: There are lymphoma cells in the lymph nodes that drain an area where skin contained lymphoma.

N2: One of the following is true:
At least 2 sets of lymph nodes from different areas contain lymphoma cells.
There are lymphoma cells in lymph nodes that do not drain areas where the skin contained lymphoma.

N3: Lymph nodes deep inside the chest or abdomen contain lymphoma cells.

### M Categories

M0: No signs of lymphoma outside of the skin or lymph nodes.

M1: Lymphoma has spread to other organs or tissues.

This system does not assign an overall stage to the lymphoma, as the system for mycosis fungoides/Sezary syndrome does. Because this system is still fairly new, it's not yet clear how well it can help predict a person's prognosis (outlook). IL-13 receptor as employed herein is a type I cytokine receptor with two subunit IL-13Rα1 (Uniprot number P78552) and IL4Rα (Q9H186). These form a dimer with IL-13. The signally occurs via activation of the JAK/Signal Transducer and Activator of Transcription (STAT) pathway.

The IL-13 receptor can also instigate IL-4 signalling.

Antagonist as employed herein refers to the reduction or inhibition of a biological activity or function, such as a physiological antagonist, in particular blocking or reducing actions of the JAK/signal Transducer and Activator of Transcription pathway.

“Antibody” as employed herein includes substantially intact antibody molecules, as well as chimeric antibodies, humanised antibodies, human antibodies (wherein at least one amino acid is mutated relative to the naturally occurring human antibodies), single chain antibodies (such as antibody heavy chains, antibody light chains), multispecific antibodies (such as bispecific antibodies), homodimers and heterodimers of antibody heavy and/or light chains, and may also include antigen binding fragments and derivatives of the same, unless the context indicates otherwise.

In one embodiment the antibody or binding fragment thereof is monoclonal.

“Antigen-binding fragment” is employed herein to refer to a functional fragment of an antibody that is capable of binding to the antigen to which it is specific. Antibody binding fragment, antigen binding fragment and binding fragment are employed interchangeably herein unless the context indicates otherwise.

Examples of antibody binding fragments include to Fab, modified Fab, Fab', modified Fab', F(ab')₂, Fv, Fab-Fv, Fab-dsFv, single domain antibodies (e.g. VH or VL or VHH), scFv, bi, tri or tetra-valent antibodies, Bis-scFv, diabodies, triabodies, tetrabodies and epitope-binding fragments of any of the above (see for example Holliger and Hudson, 2005, Nature Biotech. 23(9):1126-1136; Adair and Lawson, 2005, Drug Design Reviews-Online 2(3), 209-217). The methods for creating and manufacturing these antibody fragments are well known in the art (see for example Verma et al., 1998, Journal of Immunological Methods, 216, 165-181). Other antibody fragments for use in the present invention include the Fab and Fab' fragments described in International patent applications WO2005/003169, WO2005/003170 and WO2005/003171.

Examples of a multispecific antibody comprising a full-length antibody include a DVD-Ig, IgG-scFv, scFv-IgG, and IgG-V.
IgG-scFv as employed herein is a full-length antibody with a scFv on the C-terminal of each of the heavy chains or each of the light chains.
scFv-IgG as employed herein is a full-length antibody with a scFv on the N-terminal of each of the heavy chains or each of the light chains.
V-IgG as employed herein is a full-length antibody with a variable domain on the N-terminal of each of the heavy chains or each of the light chains.
IgG-V as employed herein is a full-length antibody with a variable domain on the C-terminal of each of the heavy chains or each of the light chains
DVD-Ig (also known as dual V domain IgG) is a fulllength antibody with 4 additional variable domains, one on the N-terminus of each heavy and each light chain.

In one embodiment the antibody binding fragment is or comprises a Fab or Fab' fragment.

Antibody binding fragments that comprise a Fab or Fab' fragment include Fabdab, Fab'dab, FabFv, Fab'Fv, FabdsFv, Fab-scFv, Fab'-scFv, Fab-(scFv)2, Fab'-(scFv)2, DiFab, DiFab'.
Fabdab as employed herein refers to a Fab fragment with a domain antibody appended to the heavy or light chain thereof, optionally via a linker.
Fab'dab as employed herein refers to a Fab' fragment with a domain antibody appended to the heavy or light chain thereof, optionally via a linker.
FabFv as employed herein refers to a Fab fragment with an additional variable region appended to the C-terminal of each of the following, the CH1 of the heavy chain and CL of the light chain see for example WO2009/040562. The format may be provided as a PEGylated version thereof see for example WO2011/061492,
Fab'Fv as employed herein is similar to FabFv, wherein the Fab portion is replaced by a Fab'. The format may be provided as a PEGylated version thereof.
FabdsFv as employed herein refers to a FabFv wherein an intra-Fv disulfide bond stabilises the appended C-terminal variable regions, see for example WO2010/035012. The format may be provided as a PEGylated version thereof
Fab-scFv (also referred to as a bibody) as employed herein is a Fab molecule with a scFv appended on the C-terminal of the light or heavy chain, optionally via a linker.
Fab'-scFv as employed herein is a Fab' molecule with a scFv appended on the C-terminal of the light or heavy chain, optionally via a linker.
DiFab as employed herein refers to two Fab molecules linked via their C-terminus of the heavy chains.
DiFab' as employed herein refers to two Fab' molecules linked via one or more disulfide bonds in the hinge region thereof.
DiFab and DiFab' molecules include chemically conjugated forms thereof.

Antibodies and binding fragments according to the present disclosure, wherein one, two or three amino acids are changed in the CDRs, refers to modified antibodies which retain the specificity to the antigen (target-antigen), i.e. which are specificity for the IL-13 receptor, in particular the IL-13Rα1 receptor.

“One, two, three amino acids changed in the CDRs” refers to no more than a total of three amino acids changed in the CDRs of a variable domain. It does not refer to up to three amino acids changes in each of the CDRs of a given variable domain.

Modified variable domains with at least 95% (such as 96, 97, 98, 99%) identity to a sequence explicitly disclosed herein form an aspect of the present disclosure. These modified variable domains retain specificity to the antigen (target-antigen), i.e. which are specific for the IL-13 receptor, in particular the IL-13Rα1 receptor.

Specific to a target antigen as employed herein refers to the fact that the antibody or binding fragment only binds the antigen to which it is specific or binds the antigen to which it is specific with greater affinity, for 2, 3, 4, 5 times greater affinity or more in comparison to the affinity to a substance or antigen to which it is non-specific.

In one embodiment the binding affinity (K_{D}) of the antibody or bindings fragments of the present disclosure is 1 nM or less, such as 0.5 nM, 0.3 nM or 0.2 nM. In one embodiment, the binding affinity is ˜254 pM.

Antibodies and binding fragments may be referred to herein as “active”.

As used herein, ‘pharmaceutical formulation’ refers to a therapeutically effective formulation comprising an antibody or binding fragment thereof and pharmaceutically acceptable diluent, excipient and/or carrier.

A ‘therapeutically effective amount’, or ‘effective amount’, or ‘therapeutically effective’, as used herein, refers to that amount which provides a therapeutic effect for a given condition and administration regimen. This is a predetermined quantity of active material calculated to produce a desired therapeutic effect in association with the required additive and diluent, i.e. a carrier or administration vehicle. Further, it is intended to mean an amount sufficient to reduce and/or prevent, a clinically significant deficit in the activity, function and response of the host/patient. Alternatively, a therapeutically effective amount is sufficient to cause an improvement in a clinically significant condition in a host/patient. As is appreciated by those skilled in the art, the amount of an active agent (such as an antibody or binding fragment according to the present disclosure) may vary depending on its specific activity. Suitable dosage amounts may contain a predetermined quantity of active composition calculated to produce the desired therapeutic effect in association with the required diluent. In the methods and use for manufacture of compositions of the invention, a therapeutically effective amount of the active component is provided, for example as a unit dose.

A therapeutically effective amount can be determined by the ordinary skilled medical or veterinary worker based on patient characteristics, such as age, weight, sex, condition, complications, other diseases, etc., as is well known in the art.

In one embodiment the patient is a human.

The term payloads as used herein includes, for example, biologically active proteins, such as enzymes, other antibody or antibody binding fragments, synthetic or naturally occurring polymers, nucleic acids and fragments thereof e.g. DNA, RNA and fragments thereof, radionuclides, particularly radioiodide, radioisotopes, chelated metals, nanoparticles and reporter groups such as fluorescent compounds or compounds which may be detected by NMR or ESR spectroscopy.

In one embodiment the payload is a toxin, for example elected from calicheamicin, aplidin, anastrozole, azacytidine, bortezomib, bryostatin-1, busulfan, combrestatins, carmustine, dolastatins, epothilones, staurosporin, maytansinoids, spongistatins, rhizoxin, halichondrins, roridins, hemiasterlins, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof.

In one embodiment the payload is a drug, for example selected from nitrogen mustard, ethylenimine derivative, alkyl sulfonates, nitrosourea, gemcitabine, triazene, folic acid analog, anthracycline, taxane, COX-2 inhibitor, pyrimidine analog, purine analog, antibiotic, enzyme inhibitor, epipodophyllotoxin, platinum coordination complex, vinca alkaloid, substituted urea, methyl hydrazine derivative, adrenocortical suppressant, hormone antagonist, endostatin, taxol, camptothecin, doxorubicin, doxorubicin analog antimetabolite, alkylating agent, antimitotic, anti-angiogenic agent, tyrosine kinase inhibitor, mTOR inhibitor, heat shock protein (HSP90) inhibitor, proteosome inhibitor, HDAC inhibitor, pro-apoptotic agent, methotrexate, CPT-11, amifostine, cisplatin, dacarbazine, dactinomycin, mechlorethamine, streptozocin, cyclophosphamide, carrnustine, lomustine, doxorubicin lipo, gemcitabine, daunorubicin, daunorubicin lipo, procarbazine, mitomycin, cytarabine, etoposide, methotrexate, 5-fluorouracil, vinblastine, vincristine, bleomycin, paclitaxel, docetaxel, aldesleukin, asparaginase, busulfan, carboplatin, cladribine, 10-hydroxy-7-ethyl-camptothecin (SN38), gefitinib, dacarbazine, floxuridine, fludarabine, hydroxyurea, ifosfamide, idarubicin, mesna, interferon alpha, interferon beta, irinotecan, mitoxantrone, topotecan, leuprolide, megestrol, melphalan, mercaptopurine, plicamycin, mitotane, pegaspargase, pentostatin, pipobroman, plicamycin, streptozocin, tamoxifen, teniposide, testolactone, thioguanine, thiotepa, uracil mustard, vinorelbine, chlorambucil aromatase inhibitors, an antimetabolites (e.g. methotrexate, 6-mercaptopurine, 6-thioguanine, cytarabine, 5-fluorouracil decarbazine), alkylating agents (e.g. mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), carboplatin, anthracyclines (e.g. daunorubicin (formerly daunomycin) and doxorubicin or doxorubicin glucuronide), antibiotics (e.g. dactinomycin (formerly actinomycin), bleomycin, mithramycin, anthramycin (AMC), calicheamicins or duocarmycins), and anti-mitotic agents (e.g. vincristine and vinblastine), an auristatin (U.S. Pat. Nos. 5,635,483; 5,780,588), for example, MMAE (monomethyl auristatin E) or MMAF (monomethyl auristatin F). In other aspects, the drug is a dolastatin or dolastatin peptidic analog or derivative, a maytansinoid. In some aspects, the maytansinoid is N 2′-deacetyl-N 2′- (3-mercapto-1-oxopropyl)-maytansine (DM1), N 2′-deacetyl-N2′-(4-mercapto-1-oxopentyl)-maytansine (DM3) or N 2′-deacetyl-N 2′(4-methyl-4-mercapto-1-oxopentyl)-maytansine (DM4). Maytansinoids are mitotic inhibitors which act by inhibiting tubulin polymerization. Maytansine was first isolated from the east African shrub Maytenus serrata (U.S. Pat. No. 3,896,111), tubulysin

Other payloads may include chelated radionuclides such as 111In and 90Y, Lu177, Bismuth213, Californium252, Iridium192 and Tungsten188/Rhenium188; or drugs such as but not limited to, alkylphosphocholines, topoisomerase I inhibitors, taxoids and suramin. Other payloads include proteins, peptides and enzymes. Enzymes of interest include, but are not limited to, proteolytic enzymes, hydrolases, lyases, isomerases, transferases. Proteins, polypeptides and peptides of interest include, but are not limited to, immunoglobulins, toxins such as abrin, ricin A, pseudomonas exotoxin, or diphtheria toxin, a protein such as insulin, tumour necrosis factor, α-interferon, β-interferon, nerve growth factor, platelet derived growth factor or tissue plasminogen activator, a thrombotic agent or an anti-angiogenic agent, e.g. angiostatin or endostatin, or, a biological response modifier such as a lymphokine, interleukin-1 (IL-1), interleukin-2 (IL-2), granulocyte macrophage colony stimulating factor (GM-CSF), granulocyte colony stimulating factor (G-CSF), nerve growth factor (NGF) or other growth factor and immunoglobulins.

Other payloads may include detectable substances useful for example in diagnosis. Examples of detectable substances include various enzymes, prosthetic groups, fluorescent materials, luminescent materials, bioluminescent materials, radioactive nuclides, positron emitting metals (for use in positron emission tomography), and nonradioactive paramagnetic metal ions. See generally U.S. Pat. No. 4,741,900 for metal ions which can be conjugated to antibodies for use as diagnostics. Suitable enzymes include horseradish peroxidase, alkaline phosphatase, beta-galactosidase, or acetylcholinesterase; suitable prosthetic groups include streptavidin, avidin and biotin; suitable fluorescent materials include umbelliferone, fluorescein, fluorescein isothiocyanate, rhodamine, dichlorotriazinylamine fluorescein, dansyl chloride and phycoerythrin; suitable luminescent materials include luminol; suitable bioluminescent materials include luciferase, luciferin, and aequorin; and suitable radioactive nuclides include 125I, 131I, 111In and 99Tc.

In another example the payload may increase the half-life of the antibody in vivo, and/or reduce immunogenicity of the antibody and/or enhance the delivery of an antibody across an epithelial barrier to the immune system. Examples of suitable effector molecules of this type include polymers, albumin, albumin binding proteins or albumin binding compounds such as those described in WO05/117984.

Where the effector molecule is a polymer it may, in general, be a synthetic or a naturally occurring polymer, for example an optionally substituted straight or branched chain polyalkylene, polyalkenylene or polyoxyalkylene polymer or a branched or unbranched polysaccharide, e.g. a homo- or hetero-polysaccharide.

Specific optional substituents which may be present on the above-mentioned synthetic polymers include one or more hydroxy, methyl or methoxy groups.

Specific examples of synthetic polymers include optionally substituted straight or branched chain poly(ethyleneglycol), poly(propyleneglycol) poly(vinylalcohol) or derivatives thereof, especially optionally substituted poly(ethyleneglycol) such as methoxypoly(ethyleneglycol) or derivatives thereof.

Specific naturally occurring polymers include lactose, amylose, dextran, glycogen or derivatives thereof.

“Derivatives” as used herein is intended to refer to modified molecules (such as polymbers) which retain the essential characteristics of the original molecule including reactive derivatives, for example thiol-selective reactive groups such as maleimides and the like. The reactive group may be linked directly or through a linker segment to the polymer. It will be appreciated that the residue of such a group will in some instances form part of the product as the linking group between the antibody fragment and the polymer.

The size of the polymer may be varied as desired but will generally be in an average molecular weight range from 500 Da to 50000 Da, for example from 5000 to 40000 Da such as from 20000 to 40000 Da. The polymer size may in particular be selected on the basis of the intended use of the product, for example ability to localize to certain tissues such as tumors or extend circulating half-life (for review see Chapman, 2002, Advanced Drug Delivery Reviews, 54, 531-545).

Thus, for example, where the product is intended to leave the circulation and penetrate tissue, for example for use in the treatment of a tumour, it may be advantageous to use a small molecular weight polymer, for example with a molecular weight of around 5000 Da. For applications where the product remains in the circulation, it may be advantageous to use a higher molecular weight polymer, for example having a molecular weight in the range from 20000 Da to 40000 Da.

Suitable polymers include a polyalkylene polymer, such as a poly(ethyleneglycol) or, especially, a methoxypoly(ethyleneglycol) or a derivative thereof, and especially with a molecular weight in the range from about 15000 Da to about 40000 Da.

In one example, antibodies for use in the present invention are attached to poly(ethyleneglycol) (PEG) moieties. In one particular example the antibody is an antibody fragment and the PEG molecules may be attached through any available amino acid side-chain or terminal amino acid functional group located in the antibody fragment, for example any free amino, imino, thiol, hydroxyl or carboxyl group. Such amino acids may occur naturally in the antibody fragment or may be engineered into the fragment using recombinant DNA methods (see for example U.S. Pat. Nos. 5,219,996; 5,667,425; WO98/25971, WO2008/038024). In one example the antibody molecule of the present invention is a modified Fab fragment wherein the modification is the addition to the C-terminal end of its heavy chain one or more amino acids to allow the attachment of an effector molecule. Suitably, the additional amino acids form a modified hinge region containing one or more cysteine residues to which the effector molecule may be attached. Multiple sites can be used to attach two or more PEG molecules.

Suitably PEG molecules are covalently linked through a thiol group of at least one cysteine residue located in the antibody fragment. Each polymer molecule attached to the modified antibody fragment may be covalently linked to the sulphur atom of a cysteine residue located in the fragment. The covalent linkage will generally be a disulphide bond or, in particular, a sulphur-carbon bond. Where a thiol group is used as the point of attachment, appropriately activated effector molecules, for example thiol selective derivatives such as maleimides and cysteine derivatives may be used. An activated polymer may be used as the starting material in the preparation of polymer-modified antibody fragments as described above. The activated polymer may be any polymer containing a thiol reactive group such as an α-halocarboxylic acid or ester, e.g. iodoacetamide, an imide, e.g. maleimide, a vinyl sulphone or a disulphide. Such starting materials may be obtained commercially (for example from Nektar, formerly Shearwater Polymers Inc., Huntsville, Ala., USA) or may be prepared from commercially available starting materials using conventional chemical procedures. Particular PEG molecules include 20K methoxy-PEG-amine (obtainable from Nektar, formerly Shearwater; Rapp Polymere; and SunBio) and M-PEG-SPA (obtainable from Nektar, formerly Shearwater).

In one embodiment, the antibody is a modified Fab fragment or diFab which is PEGylated, i.e. has PEG (poly(ethyleneglycol)) covalently attached thereto, e.g. according to the method disclosed in EP 0948544 or EP1090037 [see also “Poly(ethyleneglycol) Chemistry, Biotechnical and Biomedical Applications”, 1992, J. Milton Harris (ed), Plenum Press, NewYork, “Poly(ethyleneglycol) Chemistry and Biological Applications”, 1997, J. Milton Harris and S. Zalipsky (eds), American Chemical Society, Washington D.C. and “Bioconjugation Protein Coupling Techniques for the Biomedical Sciences”, 1998, M. Aslam and A. Dent, Grove Publishers, NewYork; Chapman, A. 2002, Advanced Drug Delivery Reviews 2002, 54:531-545]. In one example PEG is attached to a cysteine in the hinge region. In one example, a PEG modified Fab fragment has a maleimide group covalently linked to a single thiol group in a modified hinge region. A lysine residue may be covalently linked to the maleimide group and to each of the amine groups on the lysine residue may be attached a methoxypoly(ethyleneglycol) polymer having a molecular weight of approximately 20,000 Da. The total molecular weight of the PEG attached to the Fab fragment may therefore be approximately 40,000 Da.

Particular PEG molecules include 2-[3-(N-maleimido)propionamido]ethyl amide of N,N′-bis(methoxypoly(ethylene glycol) MW 20,000) modified lysine, also known as PEG2MAL40K (obtainable from Nektar, formerly Shearwater).

Alternative sources of PEG linkers include NOF who supply GL2-400MA2 (wherein m in the structure below is 5) and GL2-400MA (where m is 2) and n is approximately 450:

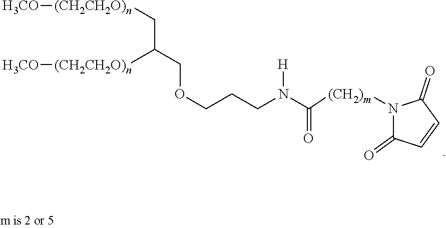

That is to say each PEG is about 20,000 Da. Further alternative PEG effector molecules of the following type:

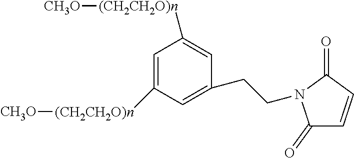

In one embodiment there is provided an antibody which is PEGylated (for example with a PEG described herein), attached through a cysteine amino acid residue at or about amino acid 226 in the chain, for example amino acid 226 of the heavy chain (by sequential numbering).

In one embodiment the antibody or binding fragment is provided as a pharmaceutical formulation comprising one or more excipients, diluents and/or carriers. Accordingly, there is provided a pharmaceutical composition comprising an antibody or binding fragment as described above.

It will be appreciated by persons skilled in the art that the antibody or antigen-binding fragment, derivative or variant thereof of the invention will generally be administered in admixture with a suitable pharmaceutical excipient diluent or carrier, selected with regard to the intended route of administration and standard pharmaceutical practice (for example, see Remington: The Science and Practice of Pharmacy, 19th edition, 1995, Ed. Alfonso Gennaro, Mack Publishing Company, Pennsylvania, USA).

For example, the antibody or antigen-binding fragment, derivative or variant thereof of the invention can be administered orally, buccally or sublingually in the form of tablets, capsules, ovules, elixirs, solutions or suspensions, which may contain flavouring or colouring agents, for immediate-, delayed- or controlled-release applications.

Such tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxy-propylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Suitable excipients in this regard include lactose, starch, cellulose, milk sugar or high molecular weight polyethylene glycols. For aqueous suspensions and/or elixirs, the compounds of the invention may be combined with various sweetening or flavouring agents, colouring matter or dyes, with emulsifying and/or suspending agents and with diluents such as water, ethanol, propylene glycol and glycerin, and combinations thereof. Alternatively, capsules may be filled with a liquid formulation.

The antibody or antigen-binding fragment, derivative or variant thereof of the invention can also be administered parenterally, for example, intravenously, intra-articularly, intra-arterially, intraperitoneally, intrathecally, intraventricularly, intrasternally, intracranially, intra-muscularly or subcutaneously, by intracavernosal injection, or they may be administered by infusion techniques. They are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well known to those skilled in the art.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets.

Example approaches: 1) Excipients such as buffers and detergents (usually Tween) are added to inhibit aggregation in aqueous formulations; 2) Freeze drying with appropriate excipients to provide bulk, stability and cosmetic appeal to the cake; 3) Formation of a glassy sugar using compounds such as trehalose.

For oral and parenteral administration, or other routes of administration, to human patients, the daily dosage level of the antibody or antigen-binding fragment, derivative or variant thereof of the invention will usually be from 1 μg to 1000 mg per adult (i.e. from about 0.015 to 15 mg/kg), administered in single or divided doses.

As an example, the dosage level may be from about 0.5 mg/kg to about 10 mg/kg, the administration regimen may be twice or three times weekly, the administration may, for example be intravenous or subcutaneous. In another embodiment the dosing regimen may be in the range once a week to once a month delivered intravenously or by subcutaneous injection.

The antibody or antigen-binding fragment, derivative or variant thereof of the invention can also be administered intranasally or by inhalation and are conveniently delivered, for example in the form of a dry powder inhaler, pump, spray or nebuliser an aerosol spray presentation from a pressurised container with the use of a suitable propellant, such as dichlorodifluoromethane, trichlorofluoro-methane, dichlorotetrafluoro-ethane, a hydrofluoroalkane such as 1,1,1,2-tetrafluoroethane (HFA 134A3 or 1,1,1,2,3,3,3-heptafluoropropane (HFA 227EA3), carbon dioxide or other suitable gas. In the case of a pressurised aerosol, the dosage unit may be determined by providing a valve to deliver a metered amount. The pressurised container, pump, spray or nebuliser may contain a solution or suspension of the active antibody or antigen-binding fragment, derivative or variant thereof, such as using a mixture of ethanol and the propellant as the solvent, which may additionally contain a lubricant, such as sorbitan trioleate. Capsules and cartridges (made, for example, from gelatin) for use in an inhaler or insufflator may be formulated to contain a powder mix of an antibody or binding fragment of the invention and a suitable powder base such as lactose or starch.

Aerosol or dry powder formulations are suitably arranged so that each dose (or metered dose or ‘puff’) contains at least 1 μg of an antibody or antigen-binding fragment, derivative or variant thereof of the invention for delivery to the patient. It will be appreciated that the overall daily dose with an aerosol will vary from patient to patient, and may be administered in a single dose or, more usually, in divided doses throughout the day.

Alternatively, the antibody or antigen-binding fragment, derivative or variant thereof of the invention can be administered in the form of a suppository or pessary, or they may be applied topically in the form of a lotion, solution, cream, ointment or dusting powder. The compounds of the invention may also be transdermally administered, for example, by the use of a skin patch. They may also be administered by the ocular route.

For ophthalmic use, the antibody or antigen-binding fragment, derivative or variant thereof of the invention can be formulated as a suspension in isotonic, pH adjusted, sterile saline, or, suitably, as solutions in isotonic, pH adjusted, sterile saline, optionally in combination with a preservative such as a benzylalkonium chloride. Alternatively, they may be formulated in an ointment such as petrolatum.

For application topically to the skin, the antibody or antigen-binding fragment, derivative or variant thereof of the invention can be formulated as a suitable ointment suspended or dissolved in, for example, a mixture with one or more of the following: mineral oil, liquid petrolatum, white petrolatum, propylene glycol, polyoxyethylene polyoxypropylene compound, emulsifying wax and water. Alternatively, the antibody or binding fragment can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

Formulations suitable for topical administration in the mouth include lozenges comprising the active ingredient in a flavoured basis, usually sucrose and acacia or tragacanth; pastilles comprising the active ingredient in an inert basis such as gelatin and glycerin, or sucrose and acacia; and mouth-washes comprising the active ingredient in a suitable liquid carrier.

In one embodiment a sustained-release drug delivery system is employed, such as microspheres. These are designed specifically to reduce the frequency of injections. An example of such a system is Nutropin Depot which encapsulates recombinant human growth hormone (rhGH) in biodegradable microspheres that, once injected, release rhGH slowly over a sustained period.

Alternatively, the antibody or antigen-binding fragment, derivative or variant thereof of the present invention can be administered by a surgically implanted device that releases the active, for example directly to the required site.

Electroporation therapy (EPT) systems can also be employed for the administration of the antibody or antigen-binding fragment, derivative or variant thereof. A device which delivers a pulsed electric field to cells increases the permeability of the cell membranes to the drug, resulting in a significant enhancement of intracellular drug delivery.

The antibody or antigen-binding fragment, derivative or variant thereof can also be delivered by electroincorporation (EI). EI occurs when small particles of up to 30 microns in diameter on the surface of the skin experience electrical pulses identical or similar to those used in electroporation. In EI, these particles are driven through the stratum corneum and into deeper layers of the skin. The particles can be loaded or coated with drugs or genes or can simply act as “bullets” that generate pores in the skin through which the drugs can enter.

An alternative method of antibody or antigen-binding fragment, derivative or variant thereof delivery is the thermo-sensitive ReGel injectable. Below body temperature, ReGel is an injectable liquid while at body temperature it immediately forms a gel reservoir that slowly erodes and dissolves into known, safe, biodegradable polymers. The active drug is delivered over time as the biopolymers dissolve.

Antibody or antigen-binding fragment, derivative or variant thereof, or pharmaceuticals can also be delivered orally. One such system employs a natural process for oral uptake of vitamin B12 in the body to co-deliver proteins and polypeptides. By employing the vitamin B12 uptake system, the protein or polypeptide can move through the intestinal wall. Complexes are produced between vitamin B12 analogues and the drug that retain both significant affinity for intrinsic factor (IF) in the vitamin B12 portion of the complex and significant bioactivity of the “antibody” portion of the complex.

In the context of this specification “comprising” is to be interpreted as “including”. Aspects of the disclosure comprising certain elements are also intended to extend to alternative embodiments “consisting” or “consisting essentially” of the relevant elements. Positively recited embodiments may be employed herein as a basis for a disclaimer. All references referred to herein are specifically incorporated by reference. Where technically appropriate, embodiments of the invention may be combined. Headings herein are employed to divide the document into sections and are not intended to be used to construe the meaning of the disclosure provided herein.

The present application claims priority. The priority documents may be used as the basis for corrections, in particular the sequences. The invention will now be described with reference to the following examples, which are merely illustrative and should not in any way be construed as limiting the scope of the present disclosure.

### EXAMPLES

### Example 1

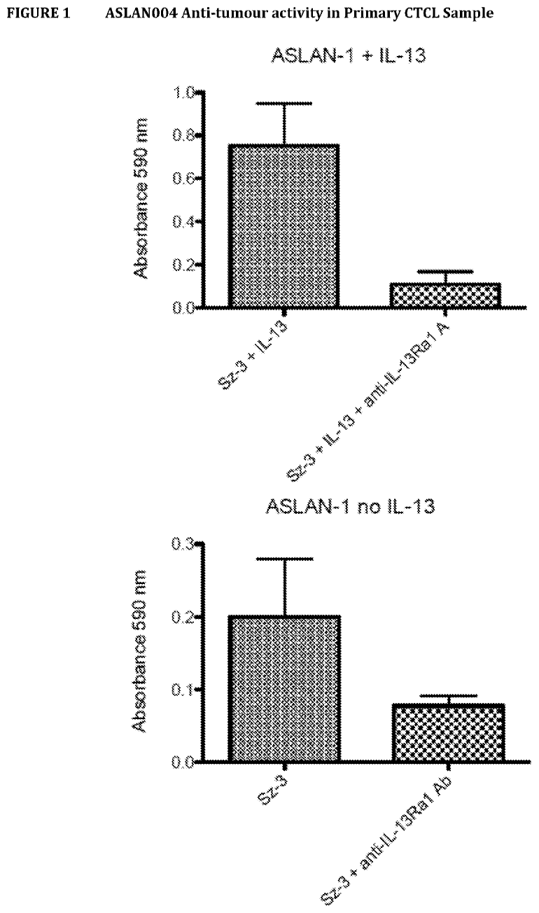

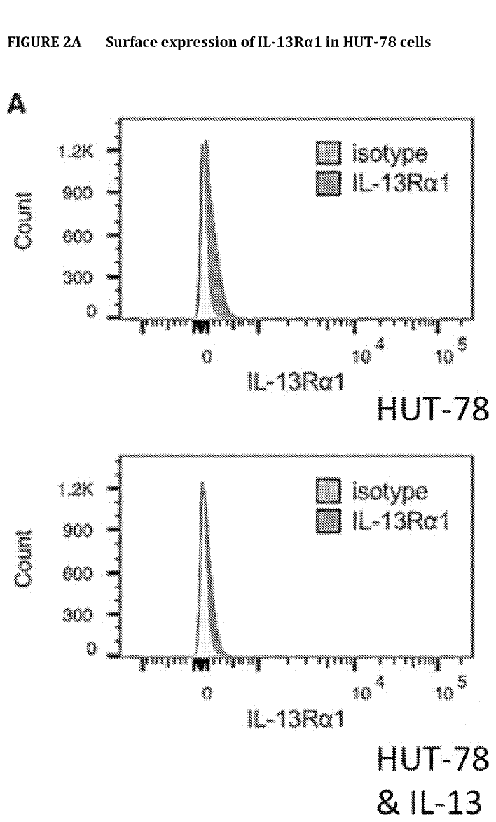

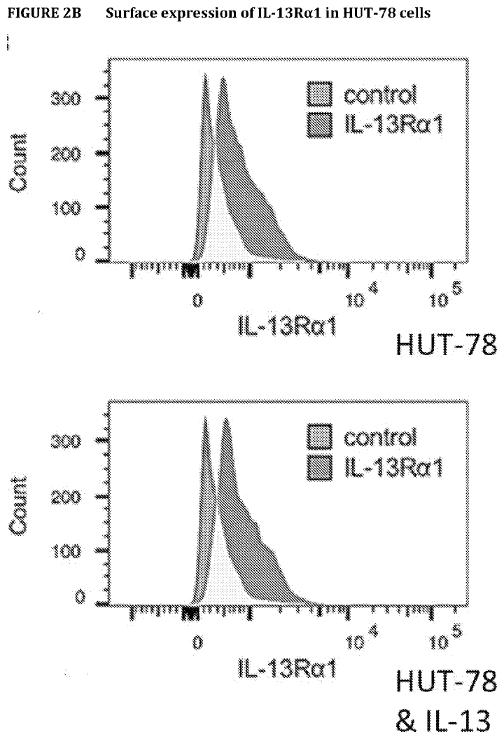

The monoclonal antibody ASLAN004 (an anti-IL-13Rα1 antibody) was tested on leukemic cells from a patient with Sézary Syndrome. In vitro the antibody concentration employed was 0.01 μg/ml. The results are shown in FIG. 1, which shows that the antibody has anti-tumor activity in the absence and also in the presence of IL-13.

### Example 2

HUT-78 have surface expression of IL-13Rα1, which can be measured by flow cytometry. Expression of IL-13Rα1 was determined by flow cytometry in Hut-78 cells treated with/without IL-13 (100 ng/ml) (dark grey histograms). We employed 2 different Abs; the anti-IL-13Rα1 from R&D (A) or from Sigma (B). As controls (grey histograms), we used the isotype in (A) and only the secondary Ab in (B).

The results show that HUT-78 cells expressed IL13Rα1 on their cell surfaces. Also, IL-13Rα1 expression does not change with or without addition of IL-13. Lastly, the results indicate a greater dynamic range with the anti-IL13Rα1 antibody from Sigma (B) vs the antibody from R&D (A).

### Example 3

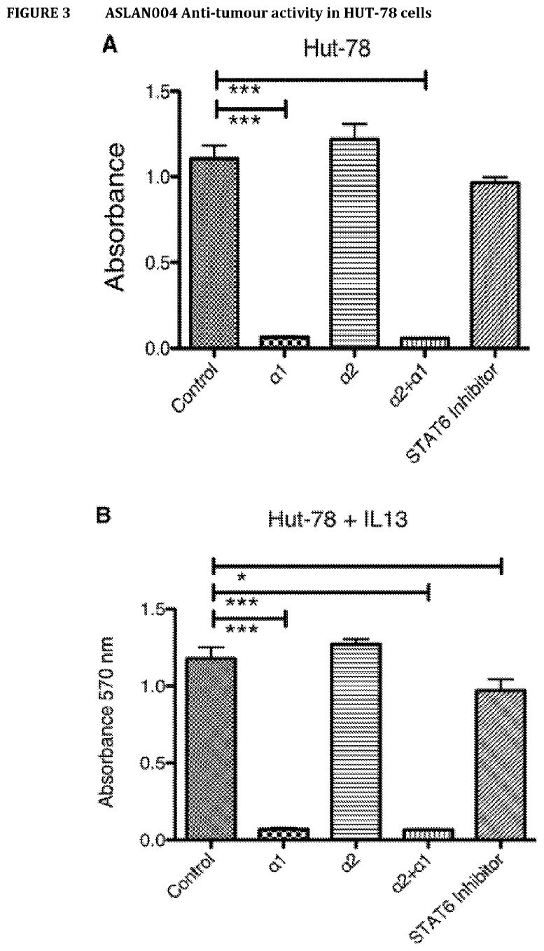

Cells were pre-incubated with inhibitors (a1=ASLAN004; a2=anti-IL-13Ra2 Ab (Biolegend); STAT-6 inhibitor=AS 1517499 (Axon) for 1 hr at 37 C followed by addition of medium alone (A) or IL-13 (B). Statistics by ANOVA followed by Tukey's post-hoc test. The results are shown in FIG. 3. ASLAN004 was a potent inhibitor of HUT-78 cell proliferation in the presence and also in the absence of IL-13.

1. A method of treating cutaneous T cell lymphoma (CTCL) comprising administering a therapeutically effective amount of an antagonist antibody or binding fragment thereof specific to the IL-13 receptor to a patient in need thereof.

2. A method according to claim 1 wherein the antibody thereof is specific to IL-13R alpha 1 (IL-13Rα1) or anti-IL13R alpha 2 (IL-13Rα2).

3. The method according to claim 2, wherein the antibody or binding fragment thereof is an anti-IL-13Rα1 antibody or binding fragment.

4. The method according to claim 3, wherein the antibody binds the epitope FFYQ.

5. The method according to claim 1, wherein the antibody or binding fragment thereof has a heavy chain variable domain comprising a CDR H1 shown in SEQ ID NO: 1, CDR H2 shown in SEQ ID NO: 2 and a CDR H3 with a sequence independently selected from SEQ ID NO: 3, 4, 12, 13, 14, 15, 16, 17, 18, 19, 20, 21, 22, 23, 24, 25, 26, 27, 28, 29, 30, 31, 32, 33, 34, 35, 36, 37 and 38, or a variable domain wherein one, two or three amino acids in the CDRs are added substituted or deleted.

6. The method according to claim 1, wherein the antibody or binding fragment thereof has a light chain variable domain comprising a CDR L1 shown in SEQ ID NO: 5, CDR L2 shown in SEQ ID NO: 6 and a CDR L3 independent selected from SEQ ID NO: 7, 39. 40, 41, 42, 43, 44, 45, 46, 47, 48, 49, 50, 51 and 52 or a variable domain wherein one, two or three amino acids in the CDRs are added substituted or deleted.

7. The method according to claim 1, wherein the antibody or binding fragment thereof has a light chain variable domain comprising a sequence shown in SEQ ID NO: 9, 56, 57, 58 or a sequence at least 95% percent identical thereto.

8. The method according to claim 1, wherein the antibody or binding fragment thereof has a light chain variable domain comprising a sequence shown in SEQ ID NO: 9.

9. The method according to claim 1, wherein the antibody or binding fragment thereof has a heavy chain variable domain comprising a sequence shown in SEQ ID NO: 8, 53, 54, 55 or a sequence at least 95% percent identical thereto.

10. The method according to claim 1, wherein the antibody or binding fragment thereof has a heavy chain variable domain comprising a sequence shown in SEQ ID NO: 8.

11. The method according to claim 1, wherein the antibody or antigen binding fragment thereof is a human or humanized antibody.

12. The method according to claim 1, wherein the antibody binding fragment, for example a fragment selected from the group consisting of an Fv, dsFv, scFv, Fab, Fab' or F(ab')₂ fragment.

13. The method according to claim 1, wherein the antibody is a full-length antibody.

14. The method according to claim 13, wherein the antibody heavy chain has the sequence shown in SEQ ID NO: 10, 59, 60, 61, 62, 63 or 64.

15. The method according to claim 13, wherein the antibody light chain has the sequence shown in SEQ ID NO: 11, 65 or 66.

16. The method according to claim 1, wherein the antibody or antigen binding fragment thereof inhibits IL-13 signaling through the IL-13 receptor complex.

17. The method according to claim 1, wherein the CTCL is refractory to first line treatment.

18. A method according to claim 1, wherein the lymphoma is mycosis fungoides.

19. The method according to claim 1, wherein the lymphoma is Sézary syndrome.

20. The method according to claim 1, wherein the antibody or binding fragment thereof is administered as a pharmaceutical formulation.

21. The method according to claim 1, wherein the anti-IL13 receptor antibody is administered at a dose in the range of 1 ng to 1000 μg.

22. (canceled)

23. The method according to claim 1, where the antibody or binding fragment is employed as part of a combination therapy comprising a further therapeutic agent.

24-25. (canceled)

26. The method according to claim 1, wherein the antibody or binding fragment is conjugated to payload.

27. The method according to claim 26, wherein the payload is a toxin or a drug molecule.

28-29. (canceled)
